(19) United States
(12) Patent Application Publication
(10) Pub. No.: US 2020/0390836 A1
(43) Pub. Date: Dec. 17, 2020

(54) SEASONED LAVER INCLUDING HEMP OIL AND MANUFACTURING METHOD THEREOF

(71) Applicant: Young-Kee LEE, Seoul (KR)

(72) Inventor: Young-Kee LEE, Seoul (KR)

(21) Appl. No.: 16/659,591

(22) Filed: Oct. 22, 2019

(30) Foreign Application Priority Data
Jun. 12, 2019 (KR) 10-2019-0069318

(51) Int. Cl. A61K 36/04; A61K 36/185

(52) U.S. Cl. CPC A61K 36/04; A61K 36/185

(57) ABSTRACT

A seasoned laver includes laver; and hemp oil applied to the laver and a manufacturing method of seasoned laver includes: a steps of preparing dry raw laver; a steps of drying the dry raw laver; a steps of first roasting to manufacture roasted laver by adding a heat source to the dry raw laver and roasting; a steps of manufacturing seasoned laver by applying hemp oil to the roasted laver; and a steps of second roasting to apply a heat source to the seasoned laver.

### TECHNICAL FIELD

The present invention relates to laver.

### Technical Background of the Invention

Generally, laver is also known as “green laver” and “purple laver”, is a seaweed from the family Bangiaceae and order Bangiales in the division Rhodophyta.

Laver not only includes high-quality protein and fibre, and nutrients such as minerals and vitamins that are essential to our bodies but are easily deficient, but also it is the strongest alkaline food among foods, and is known as an ideal health food that provides nutritional balance to modern people who have low calories and severe calorie intake and nutritional imbalance.

Furthermore, laver is one of the most preferred foods among seaweeds because of its taste and aroma. In addition to being used as the main material for Gimbap (Dried Seaweed Rolls), crushed laver is used as a garnish of Dumpling Soup, noodles, Udon, porridge, etc. It is also widely used to make fried rice, Bibimbap and riceballs. Thus, laver is attracting attention as a food rich in nutrients.

However, since the existing laver is simply dried and provided, there is a problem that it does not meet the taste of health-oriented consumers.

### SUMMARY OF THE INVENTION

### Technical Problem

An object of the present invention is to provide seasoned laver that can satisfy the tastes of health-oriented consumers, including improving the heart and cardiovascular functions by adding omega-3, 6, and 9 ingredients, Essential Fatty Acids of hemp oil, and a manufacturing method thereof.

Another object of the present invention is to provide the seasoned laver that can be roasted at temperatures below the smoke point after hemp oil is applied to prevent the formation of obnoxious odours and harmful ingredients due to the denaturation of oil at high temperatures and its manufacturing method.

An object of the present invention is to provide the seasoned laver that has a unique savour and flavour and can enhance the taste and nutrition and minimize destruction of nutrients with dry flakes such as dried anchovies, dried shrimp, and dried vegetables and its manufacturing method.

The object of the present invention is not limited to the above-mentioned objects, and other objects not mentioned will be clearly understood by any person skilled in the relevant field of technology from the following description.

### Means for Solving Problems

To achieve the above objects, the seasoned laver according to an embodiment of the present invention includes laver; hemp oil applied to the laver.

The seasoned laver according to the embodiment of the present invention described above may further include an additive includng salt added to the laver; dry flakes added to the laver and including dried anchovies, dried shrimp, and dried vegetables.

The laver may be roasted laver.

Meanwhile, a manufacturing method of the seasoned laver according to another embodiment of the present invention includes the steps of: preparing dry raw laver; drying the dry raw laver; first roasting to manufacture roasted laver by adding a heat source to the dry raw laver and roasting; manufacturing seasoned laver by applying hemp oil to the roasted laver; and second roasting to apply a heat source to the seasoned laver.

In the step of second roasting, the seasoned laver can be heated in a temperature range of 170° C. to 200° C.

In the manufacturing of the seasoned laver, to the seasoned laver with the hemp oil, an additive including salt and dry flakes including dried anchovies, dried shrimp and dried vegetables may be further added.

In the step of drying, the dehydrated raw laver may be dried in a temperature range of 210° C. to 230° C.

In the step of first roasting, the dry raw laver can be heated in a temperature range of 250° C. to 280° C.

The step of preparing the dry raw laver may include the steps of: cutting raw material of the gathered laver; manufacturing raw laver by washing the raw material of the cutted laver; and dehydrating the washed raw laver to dry raw laver.

### Effects of the invention

As described above, the seasoned laver and its manufacturing method according to the embodiments of the present invention may have the following effects.

According to the embodiments of the present invention, since a technical element including laver and hemp oil is provided, omega-3, 6, and 9 ingredients Essential Fatty Acids of hemp oil may be added to the laver. In this way, health-oriented functionality that plays a key role in maintaining and activating the body's function, such as improving heart and cardiovascular function, can be reinforced. In particular, hemp oil includes minerals such as calcium, iron, magnesium, manganese, phosphorus, potassium, zinc and vitamins B1, B2, B3, B6, D and E, and phytosterol of hemp oil can reduce total serum cholesterol by an average of 10% and reduce bad LDL cholesterol by an average of 13%. Further, hemp oil includes 9 essential amino acids and 18 amino acids, GLA (γ-linolenic acid), vitamins, minerals and dietary fibre. Aarginine, which helps fat burning and vasodilation, is also includeed four times as much as eel and 15 times as much as salmon, and many degenerative diseases due to aging can be prevented by taking the seasoned laver of the present invention. Further, hemp oil has been known to have anti-cancer and antioxidant properties, and is rich in tocopherol, an antioxidant known to be effective in arteriosclerosis and phytol, as well as in Alzheimer's. Therefore, the seasoned laver of the present invention can play a sufficient role as a functional food which can have nutritional significance and physiological effects in favour of diseases and prevention.

Further, according to the embodiments of the present invention, since the technical element in which seasoned laver with hemp oil is further added an additive including salt, and dry flakes including dried anchovies, dried shrimp and dried vegetables, is provided, it has a unique savour and flavour of laver, and can be improved in taste and nutrition with dry flakes such as dried anchovies, dried shrimp, dried vegetables, etc., and can be minimized destruction of nutrients.

Further, according to another embodiment of the present invention, since the technical element for heating the seasoned laver in a temperature range of 170° C. to 200° C. after applying the hemp oil is provided, roasting may proceed at a temperature below the smoke point of the hemp oil, thereby preventing the formation of obnoxious odours and harmful ingredients due to the denaturation of oil at high temperatures.

Further, according to another embodiment of the present invention, after applying hemp oil to the dried laver, and adding an additive including salt, and dry flakes including dried anchovies, dried shrimp and dried vegetables, seasoned laver is manufactured by roasting at a temperature below the smoke point. Therefore, the formation of obnoxious odours and harmful ingredients due to the denaturation of oil at high temperature can be prevented, and it has a unique savour and flavour of the seasoned laver, with cold press extraction, the destruction of nutrients in hemp oil can be minimized to meet the preferences of health oriented consumers.

Further, according to embodiments of the present invention, not limited to the above-mentioned effects, other effects that are not mentioned will be clearly understood by the person skilled in the relevant field of technology from the following description.

### BRIEF DESCRIPTION OF DRAWINGS

FIG. 1 is a view schematically showing seasoned laver according to an embodiment of the present invention.

FIG. 2 is a flowchart schematically illustrating the manufacturing method of the seasoned laver of FIG. 1.

FIG. 3 is a flowchart illustrating a preparation process of the dry raw laver.

### DETAILED DESCRIPTION OF THE PREFERRED EMBODIMENT

Hereinafter, embodiments of the present invention will be described in detail with reference to the accompanying drawings so that a person skilled in the art may easily execute the present invention. However, the invention can be executed in various different forms and is not limited to the embodiments described herein.

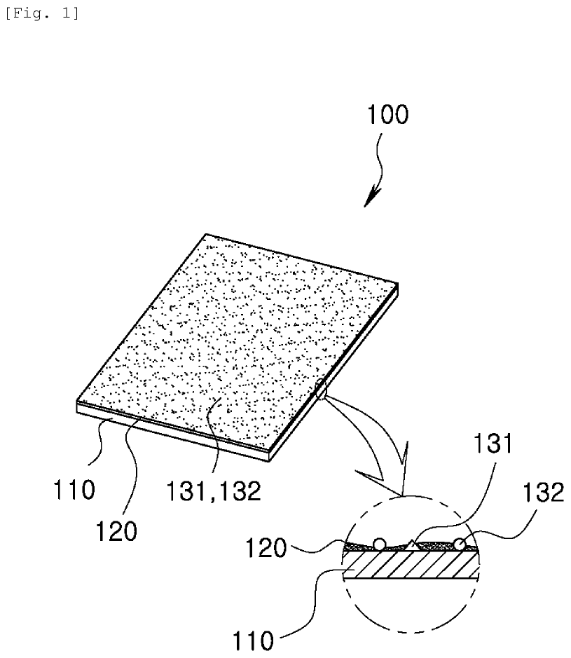

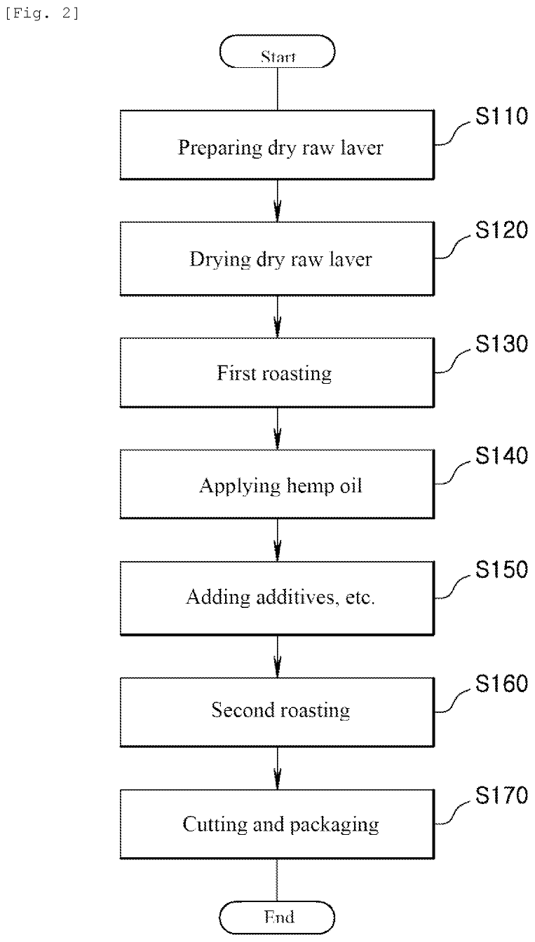

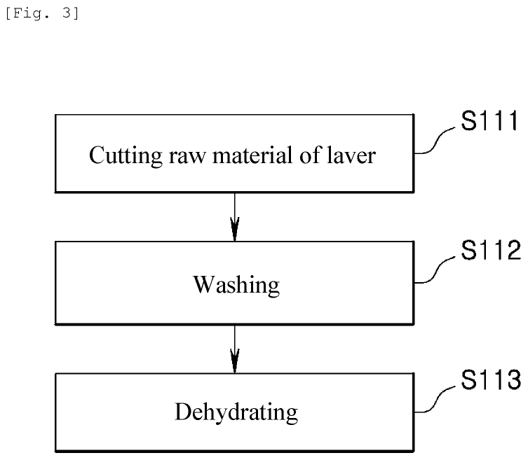

FIG. 1 is a view schematically showing seasoned laver according to an embodiment of the present invention, FIG. 2 is a flowchart schematically illustrating the manufacturing method of the seasoned laver of FIG. 1, and FIG. 3 is a flowchart illustrating a preparation process of the dry raw laver.

As shown in FIGS. 1 to 3, seasoned laver 100 according to an embodiment of the present invention includes laver 110 and hemp oil 120. Hereinafter, each element will be described in detail with reference to FIGS. 1 to 3.

The laver 110 is an element constituting the base of the seasoned laver of the present invention, as shown in FIG. 1. This laver 110 can be roasted by cutting raw material of the gathered laver into a set size, washing the raw material of the cutted laver to manufacture the raw laver, dehydrating the washed raw laver, drying the dehydrated raw laver, and applying a heat source to the dry raw laver, as shown in FIGS. 2 and 3.

Hemp oil 120 is an element for providing consumers with health-oriented functionality that plays a key role in maintaining and activating the immune system of the body. This hemp oil 120, as shown in FIGS. 1 and 2, may be applied to the laver (for example, roasted laver 110).

Therefore, since the above elements are provided, omega-3, 6, and 9 ingredients Essential Fatty Acids of hemp oil [Oil extracted from raw hemp seed at 40° C. or lower by cold pressed technique without roasting] 120 may be added to the laver. In this way, health-oriented functionality that plays a key role in maintaining and activating the body's function, such as improving heart and cardiovascular function, can be reinforced. In particular, hemp oil 120 includes minerals such as calcium, iron, magnesium, manganese, phosphorus, potassium, zinc and vitamins B1, B2, B3, B6, D and E, and phytosterol of hemp oil 120 can reduce total serum cholesterol by an average of 10% and reduce bad LDL cholesterol by an average of 13%. Further, hemp oil 120 includes 9 essential amino acids and 18 amino acids, GLA (γ-linolenic acid), vitamins, minerals and dietary fibre. Aarginine, which helps fat burning and vasodilation, is also included four times as much as eel and 15 times as much as salmon, and many degenerative diseases due to aging can be prevented by taking the seasoned laver of the present invention. Further, hemp oil 120 has been known to have anti-cancer and antioxidant properties, and is rich in tocopherol, an antioxidant known to be effective in arteriosclerosis and phytol, as well as in Alzheimer's. Therefore, the seasoned laver of the present invention can play a sufficient role as a functional food which can have nutritional significance and physiological effects in favour of diseases and prevention.

In addition, the seasoned laver 100 according to an embodiment of the present invention, as shown in FIGS. 1 and 2, may further include an additive 131 added to the laver 110 to which the hemp oil 120 is applied, and dry flake 132. The additive 131 may include salt, and the dry flake 131 may include dried anchovy, dried shrimp, and dried vegetables. Thus, there is a unique savour and flavour, and the dry flakes 132 such as dried anchovy, dried shrimp, dried vegetables, etc. can enhance the taste and nutrition, and the destruction of nutrients can be minimized.

Hereinafter, referring to FIG. 1 and FIG. 2 continuously, a manufacturing method of the seasoned laver 100 according to another embodiment of the present invention will be described.

First, prepare dry raw laver S110.

Specifically, as shown in FIG. 3, the process of preparing dry raw laver S110 is: cutting raw material of the gathered laver into a set size S111, washing the raw material of the cutted laver to manufacture the raw laver S112, dehydrating the washed raw laver to dry raw laver S113.

More specifically, the preparation process S110 of the dry raw laver prepares the dry raw laver by using the raw material of laver. Generally, raw material of laver is produced from October to April every year and can be easily changed after harvesting, so it is usually processed as dry raw laver. The raw material of laver is red when it changes, and in this state, the pigment protein contained in a large amount of raw material of laver is lost and is not available.

After harvesting the raw material of the laver, dry raw laver in a dried form can be produced by natural drying or the like. For example, after harvesting raw material of laver, the raw material of laver is washed and dehydrated in purified salt water having a salinity of 5 to 7% and a temperature of 40° C. to 45° C., and dried for 20 to 40 hours in the shade of a temperature of 15° C. to 20° C., thereby producing dry raw laver.

In particular, in the present invention by washing the raw material of the laver using purified salt water having the above-described salinity, fine impurities that may remain inside the raw material of the laver by osmotic action can be easily discharged to the outside. Meanwhile, dry raw laver, manufactured in the preparation process S110 of dry raw laver, means a plate-type laver, which is manufactured after washing, dehydrating, and washing the raw material of laver, and then placed in a booboo blind with a certain thickness and width, and then through the drying process.

Then, dring dry raw laver S120.

Specifically, a drying process S120 is a process of manufacturing a dry raw laver by removing the moisture present in the dry raw laver. In the drying process S120, by using an infrared ray or a heater to remove the moisture present in the dry raw laver, it is possible to manufacture dry raw laver.

In particular, in the drying process S120 by drying the dry raw laver in a temperature range of 210° C. to 230° C., it is possible to remove the moisture present in the dry raw laver. As a result of the experiment, it was confirmed that the moisture inside the dry raw laver was not sufficiently removed when drying dry raw laver was performed in the drying process S120 below 210° C., and when drying raw laver was performed exceeding 230° C., it was confirmed that the surface of the dry raw laver was deformed by sudden excessive heat. Therefore, the drying process S120 is preferably carried out in the first in a temperature range of 210° C. to 230° C.

Thereafter, a first roasting S130.

Specifically, the first roasting process S130 is a process of manufacturing roasted laver by adding a heat source to the dry raw laver and roasting. The first roasting process S130 can manufacture the roasted laver by heating the drying raw laver in a temperature range of 250° C. to 280° C. By passing through the first roasting process S1300, the roasted laver has a colour changed to blue-green, and moisture remaining in the drying raw laver can be removed.

In particular, in the first roasting process S130, the dried raw laver can be roasted by heating the dry raw laver that is not seasoned to a temperature range of 250° C. to 280° C. As a result of the experiment, it was confirmed that the dry raw laver was not roasted sufficiently when the roasting of the dry raw laver was performed at the first roasting process S130 below 250° C., and when the roasting was performed exceeding 280° C., the dry raw laver was burned by sudden excessive heat. Therefore, the first roasting process S130 is preferably carried out in a temperature range of 250° C. to 280° C.

Then, apply the hemp oil S140.

Specifically, the applying process S140 of the hemp oil is a process of manufacturing seasoned laver by applying the hemp oil to the roasted laver. In the applying process of the hemp oil S140, by applying the hemp oil to the roasted laver, it is possible to enhance the flavour of the seasoned laver to be manufactured.

Here, the raw hemp seed oil extracted by cold pressing is used as the hemp oil without roasting the raw hemp seed. For reference, the hemp seed oil is replaced with at least one vegetable oil selected from the group consisting of sesame oil, Perilla oil, Schizandra, olive oil, soybean oil, corn seed oil, corn oil, grape seed oil, sunflower seed oil, canola oil, safflower seed oil and cottonseed oil used as the existing oil.

By applying hemp oil to the roasted laver, it is possible to enhance the useful ingredients of the hemp oil and the flavour of the seasoned laver. As the temperature for expressing oil is extracted below 40° C. by cold press technique, useful ingredients in the hemp oil can be protected.

Thereafter, additives and the like are added for additional seasoning S150.

Specifically, the adding process S150 is a process of adding, mixing and seasoning the additives containing salt and the dry flakes including dried anchovies, dried shrimp and dried vegetables to the roasted laver applied with hemp oil. In the adding process S150, hemp seed powder may be further added together with the dry flake.

For example, in the addition process S150, when the hemp oil is applied roasted laver, it is 100% by weight, by adding 0.5% to 2% by weight of salt, the roasted laver can be seasoned, and by adding 1% to 5% by weight of dried flake including dried anchovies, dried shrimp, and dried vegetables, the taste and nutrition can be enhanced to increase consumer preference. By adding more hemp seed powder to the roasted laver, the taste and nutrients can be enhanced to increase consumer preference. Further, in the present invention, salt from deep sea water rich in minerals may be used as the salt used in the adding process S150.

Meanwhile, in the adding process S150, an additive including salt and dry flakes including dried anchovy, dried shrimp, and dried vegetables are added to prepare roasted laver, as an example, but the technical idea of the present invention is not limited to the above example, and in addition to the dry flakes including the additive including salt and dried anchovies, dried shrimp, and dried vegetables, according to consumers' preferences and manufacturing purposes, the application amount, content, additives, etc. may be variously modified, and these modifications are included in the scope of a right of the technical idea of the present invention.

Next, a second roasting S160.

Specifically, the second roasting process S160 is a process of manufacturing seasoned laver by adding a heat source to the seasoned laver and roasting. In the second roasting process S160, the seasoned laver can be manufactured by heating the seasoned laver in a temperature range of 170° C. to 200° C. which is a temperature below the smoke point of the oil. By going through the second roasting process at a temperature below the smoke point as described above, no harmful ingredients such as benzopyrene are produced, preventing the occurrence of an obnoxious odour due to the denaturation of oil at high temperature, thereby producing seasoned laver with savoury flavour.

For reference, in the manufacture of seasoned laver, in the case of existing technologies for heating oiled laver above the smoke point, carcinogens such as benzopyrene or harmful ingredients to human body such as trans fats, active oxygen, and peroxides may be produced by denaturation of oil ingredients, and in the present invention, the formation of such harmful ingredients can be prevented.

As a result of the experiment, it was confirmed that the hemp oil does not permeate sufficiently in the second roasting process S160 when the roasting of the seasoned laver is performed below 170° C., and that harmful ingredients are generated when the roasting is performed exceeding 200° C. Therefore, the second roasting process S160 is preferably carried out in a temperature range of 190° C. to 200° C.

Finally, the cutting and packaging S170.

Specifically, the cutting and packaging process S170 is a process of cutting the seasoned laver by a certain unit, then packaging and commercializing. In the cutting and packaging process S170, the element for cutting the seasoned laver, then packaging and commercializing, may be variously modified according to the purpose of the manufacturer or user, and since the elements of such commercialization are prior art, for convenience and clarity of description of the technical idea of the present invention, a detailed description will be omitted.

Hereinafter, the manufacturing method of seasoned laver according to another embodiment of the present invention will be described in more detail.

### Embodiment 1

First, the raw material of the laver was prepared, then it was washed in 6% salt and purified salt water at a temperature of 43° C., then dehydrated and dried for 30 hours in a shade of 18° C. to manufacture dry raw laver.

Next, the dry raw laver was dried at a temperature of 220° C., and then roasted laver was manufactured by heating it at a temperature of 260° C.

Next, the roasted laver was seasoned by applying salt, adding dry flakes including dried anchovies, dried shrimp and dried vegetables, and roasting the roasted laver to which hemp oil was applied at a temperature of 180° C., the seasoned laver with hemp oil was manufactured.

### Embodiment 2

In the same manner as in Embodiment 1, seasoned laver was manufactured, but in embodiment 2, salt made from deep sea water was used.

### Embodiment 3

In the same manner as in Embodiment 1, seasoned laver was manufactured, but, in embodiment 3, the roasted laver was manufactured by drying dry raw laver at a temperature of 240° C. and then heating at a temperature of 270° C., the seasoned laver was manufactured by roasting the roasted laver with hemp oil, salt and dry flakes at a temperature of 190° C.

### Embodiment 4

In the same manner as in Embodiment 1, seasoned laver was manufactured, but, in Embodiment 4, the roasted laver was manufactured by drying the dry raw laver at a temperature of 240° C. and then at a temperature of 270° C., and as the salt, salt made from deep sea water was used.

### Comparative Example 1

After applying hemp oil as in the prior art, the seasoned laver was manufactured by heating at high temperature, but, in comparative example 1, the dry raw laver manufactured according to Embodiment 1 was dried at a temperature of 220° C., and then deep sea salt was applied. Thereafter, the laver to which the hemp oil and the deep sea water salt were applied was heated at a temperature of 360° C., thereby to manufacture roasted laver.

### Comparative example 2

Commercially available seasoned laver (manufactured by C) was prepared and used as seasoned laver according to comparative example 2.

<Sensory Evaluation >

As described above, the sensory evaluation on the taste, savour, preference, etc. of the seasoned laver manufactured according to Embodiments 1, 2, 3 and 4 and the seasoned laver manufactured according to comparative examples 1 and 2, and the result was shown to the following [Table 2]. Sensory evaluation was conducted on 25 general consumers, and the score and evaluation standard were 9-point scoring method, and are shown in [Table 1] below.

TABLE 1

| Score | Evaluation standard |
|---|---|
| 9 | Excellent |
| 7 | Good |
| 5 | Average |
| 3 | Poor |
| 1 | Terrible |

TABLE 2

| | Taste | Savour | Appearance | Overall |
|---|---|---|---|---|
| Classification | (flavour) | (smell) | (colour) | preference |
| Embodiment 1 | 7.9 | 7.9 | 7.9 | 7.9 |
| Embodiment 2 | 7.7 | 7.6 | 7.6 | 7.6 |
| Embodiment 3 | 7.9 | 7.9 | 7.9 | 7.9 |
| Embodiment 4 | 7.7 | 7.7 | 7.7 | 7.7 |
| Comparative | 5.2 | 5.1 | 5.0 | 5.1 |
| example 1 | | | | |
| Comparative | 7.2 | 7.1 | 7.3 | 7.2 |
| example 2 | | | | |

As shown in [Table 2], in the results of comparing the taste, savour, appearance and overall preference of the seasoned laver manufactured according to the Embodiments 1, 2, 3, and 4 and the seasoned laver according to the comparative examples 1 and 2, it was found that the seasoned laver manufactured by the Embodiments was superior in overall taste (flavour), savour (smell) and appearance (colour) to the seasoned laver according to comparative examples.

As in the embodiment, the seasoned laver was manufactured by applying hemp oil, an additive including salt and dry flakes including dried anchovies, dried shrimp and dried vegetables, by seasoning and by roasting at a temperature below the smoke point. Therefore, the flavour of the seasoned laver can be prevented from being inhibited by the denaturation of oil components at high temperature, and it is judged that the dry flakes satisfy the taste of consumers by enhancing the taste and nutrition of the seasoned laver.

<Content Analysis of Benzopyrene>

Benzopyrene is a type of polycyclic aromatic hydrocarbon (PAH), which is an environmental hormone that causes various cancers and causes mutations when accumulated in the human body. When the food is cooked or processed at a high temperature, carbohydrates, proteins, fats, and the like, which are the main components of the food, may be produced by incomplete combustion.

The content of benzopyrene was studied by the experimental method announced by the Ministry of Food and Drug Safety, and as a test equipment, high speed liquid chromatography Younglin instrument (MODEL: SP925S) was used as a fluorescent detector, JASCO FP-2020PLUS autosampler as a fluorescent detector. As an internal material, 3-methylcholaterene was used, extracted with N, N-dimethylformamide-water (9:1) and hexane and purified by SPE (Solid Phase Extraction) cartridge; the analysis was performed with the high speed liquid chromatography/fluorescence detector.

TABLE 3

| Classification | Content analysis of benzopyrene (ppb) |
|---|---|
| Embodiment 1 | Non-detection |
| Embodiment 2 | Non-detection |
| Embodiment 3 | Non-detection |
| Embodiment 4 | Non-detection |
| Comparative example 1 | 2.3 |

Referring to [Table 3], for the seasoned laver manufactured according to Embodiments 1, 2, 3, and 4, benzopyrene was confirmed to be 2.0 ppb or less, the legal standard value. The unseasoned dry laver is heated at high temperature, after which hemp oil is applied and seasoned with additives including deep sea water salt and dry flakes including dried anchovies, dried shrimp and dried vegetables, and by roasting it again at a low temperature below the smoke point of hemp oil to manufacture seasoned laver. Through such a process, it was found that various foreign substances and benzopyrene, a primary carcinogen, were reduced.

Although a preferred embodiment of the present invention has been described in detail above, the scope of a right of the present invention is not limited thereto, and many variations and modifications of a person skilled in the relevant field of technology using the basic concepts of the invention as defined in the following claims are also within the scope of a right of the invention.

### REFERENCE SIGNS LIST

100: seasoned laver

110: laver

120: hemp oil

131: additive (salt, etc.)

132: dry flake (dried anchovies, dried shrimp, and dried vegetables etc.)

What is claimed is:

1. Seasoned laver comprising:
laver; and
hemp oil applied to the laver.

2. The seasoned laver of claim 1, further comprising:
an additive includng salt added to the laver; and
an dry flake added to the laver and including dried anchovies, dried shrimp, and dried vegetables.

3. A manufacturing method of seasoned laver comprising:
a steps of preparing dry raw laver;
a steps of drying the dry raw laver;
a steps of first roasting to manufacture roasted laver by adding a heat source to the dry raw laver and roasting;
a steps of manufacturing seasoned laver by applying hemp oil to the roasted laver; and
a steps of second roasting to apply a heat source to the seasoned laver.
